(19) United States
(12) Patent Application Publication
(10) Pub. No.: US 2004/0171802 A1
(43) Pub. Date: Sep. 2, 2004

(54) Haemophilus influenzae antigens and corresponding dna fragments

(76) Inventors: Josee Hamel, Sillery (CA); France Couture, St.-Ferreol-les-neiges (CA); Bernard R. Brodeur, Sillery (CA); Denis Martin, St-Augustin-de-Desmaures (FR); Catherine Quellet, St-Jean-Chrysostome (CA); Mireille Tremblay, Quebec (CA); Annie Charbonneau, Quebec (CA); Catherine Vayssier, Sillery (CA)

Correspondence Address: McDermott Will & Emery, 600 13th Street NW, Washington, DC, 20005-3096, US

(21) Appl. No.: 10/398,186

(22) PCT Filed: Oct. 2, 2001
(86) PCT No.: PCT/CA01/01402

Related U.S. Application Data
(60) Provisional application No. 60/236,712, filed on Oct. 2, 2000.

(51) Int. Cl. C07K 14/195; C12N 1/21; C07H 21/04

(52) U.S. Cl. 530/350; 435/69.3; 435/320.1; 435/252.3; 536/23.7

(57) ABSTRACT

The present invention relates to polypeptides of Haemophilus influenzae which may be used for prophylaxis, diagnostic and/or therapy purposes.

### FIELD OF THE INVENTION

The present invention is related to polypeptides of Haemophilus influenzae and corresponding DNA fragments, which may be useful to prevent, diagnose and/or treat Haemophilus influenzae infections in individuals such as humans.

### BACKGROUND OF THE INVENTION

Haemophilus influenzae is a Gram-negative rod that is found in nature only as a human pathogen. Isolates of H. influenzae can be subdivided into encapsulated and non-encapsulated forms. Encapsulated strains express one of six structurally and antigenically distinct capsular polysacchariaes that are designated, types “a” to “f”. Non-encapsulated strains are defined by their failure to agglutinate with antisera against the recognised H. influenzae capsular polysaccharides and are referred to as nontypeable.

Nontypeable H. influenzae strains commonly colonise the upper respiratory tract, including the nasopharynx and the posterior oropharynx. A number of surveys of healthy individuals indicate colonisation rates between 40% to 80% among both children and adults (Spinola S. M., Peacock J., Denny F. W., Smith D. L. and Cannon J. G. (1986) Epidemiology of colonisation by nontypeable Haemophilus influenzae in children: a longitudinal study. J. Infect. Dis. 154:100-109; Trottier S., Stenberg K. and Svanborg-Eden C. (1989) Turn over of nontypeable Haemophilus influenzae in the nasopharynges of healthy children. J. Clin. Microbiol. 27:2175-2179). Colonisation with a particular strain may persist for weeks or months with most individuals by remaining asymptomatic throughout this period. The pathogenesis of disease due to nontypeable H. influenzae involves contiguous spread within the respiratory tract. Spread to adjacent areas is usually a consequence of abnormalities in either non-specific or specific host defences. So, nontypeable H. influenzae causes a variety of respiratory tract infections in children and adults including otitis, sinusitis, bronchitis and pneumonia. These infections may become chronic or recurrent in patients with bronchitis or otitis. In fact, in infants and children, nontypeable H. influenzae is a frequent cause of acute otitis media and is commonly implicated in recurrent otitis media (Harabuchi Y., Fadden H., Yamanaka N., Duffy L., Wolf J., Krystofik D. and Tonawanda/Williamsville Pediatrics. (1994) Nasopharyngeal colonisation with nontypeable Haemophilus influenzae and recurrent otitis media. J. Infect. Dis. 170:862-866). In infants, nontypeable H. influenzae is responsible for between 27% and 37% of the first episode of otitis media by the age of 1 year (Smith-Vaughan H. C., Sriprakash K. S., Mathews J. D. and Kemp D. J. (1997) Nonencapsulated Haemophilus influenzae in aboriginal infants with otitis media: prolonged carriage of P2 porin variants and evidence for horizontal P2 gene transfer.

Infect. Immun. 65:1468-1474; Teele D. W., Klein J. O., Rosner B. and Greater Boston Otitis Media study Group. (1989) Epidemiology of otitis media during the first seven years of life in children in Greater Boston: a prospective, cohort study. J. Infect. Dis. 160:83-94). Meningitis is sometimes caused by nontypeable H. influenzae and accounts for 1-3% of all cases. But, nontypeable H. influenzae is particularly prevalent in hosts with an underlying disease which affects the innate mucosal immune system, such as chronic obstructive pulmonary disease (COPD) and cystic fibrosis (Murphy T. F. and Sethi S. (1992) Bacterial infection in chronic obstructive pulmonary disease. Am. Rev. Respir. Dis. 146:1067-1083; St. Geme J. W. III. (1993) Nontypeable Haemophilus influenzae disease: epidemiology, pathogenesis and prospects for prevention. Infect. Agents Dis. 2:1-16). Nontypeable H. influenzae strains are found predominantly during exacerbations when the sputum becomes mucopurulent. Acute infective exacerbations of chronic bronchitis play an important role in the morbidity and mortality of patients with chronic pulmonary diseases.

The etiologies of community-acquired pneumonia appear to have changed in the last decade. While Streptococcus pneumoniae remains the predominant pathogen, the proportion of cases involving other organisms has increased. H. influenzae is now often reported as being the second most common cause of pneumonia. Ten percent of H. influenzae pneumoniae cases develop into bacteremia. In developing countries pneumonia caused by nontypeable H. influenzae is apparently an important cause of morbidity and mortality in children.

Although several H. influenzae type b polysaccharide conjugated vaccines have been developed, these vaccines are ineffective against disease caused by other H. influenzae strains (Scheifele D. W., Jadavji T. P., Law B. J., Gold R., Macdonald N. E., Lebel M. H., Mills E. L., Dery P., Halperin S. A., Morris R. F., Marchessault V. and Duclos P. J. (1996) Recent trends in pediatric Haemophilus influenzae type b infections in Canada. Can. Med. Assoc. J. 154:1041-1047; Schulte E. E., Birkhead G. S., Kondracki S. F. and Morse D. L. (1994) Patterns of Haemophilus influenzae type b invasive disease in New York State, 1987-991 the role of vaccination requirements for day-care attendance. Pediatrics 94:1014-1016). The identification of conserved cross-protective antigens is critical for the development of a universal vaccine against H. influenzae infection and disease. Outer membrane proteins such as P1, P2, P4, P6, PCP, OMP26 and D-15 have been identified or are currently explored as potential vaccine antigens (Foxwell A. R., Kyd J. M. and Cripps A. W. (1998) Nontypeable Haemophilus influenzae: Pathogenesis and Prevention. Microbiol. Mol. Biol. Rev. 62:294-308). Protein D-15 is the only conserved immunogen that has been described, in the scientific literature, as being capable of conferring protection against multiple serotypes and nontypeable strains.

Therefore there remains an unmet need for H. influenzae polypeptides that may be useful to prevent, diagnose and/or treat Haemophilus influenzae infections in individuals such as humans.

### SUMMARY OF THE INVENTION

According to one aspect, the present invention provides an isolated polynucleotide encoding a polypeptide having at least 70% identity to a second polypeptide comprising a sequence chosen from SEQ ID Nos: 2, 4, 6, 8, 10, 12, 14, 16, 18, 20, 22 and 24 or fragments or analogs thereof.

According to one aspect, the present invention relates to polypeptides which comprise an amino acid sequence selected from SEQ ID Nos: 2, 4, 6, 8, 10, 12, 14, 16, 18, 20, 22 and 24 or fragments or analogs thereof.

In other aspects, there are provided polypeptides encoded by polynucleotides of the invention, pharmaceutical compositions, vectors comprising polynucleotides of the invention operably linked to an expression control region, as well as host cells transfected with said vectors and processes of producing polypeptides comprising culturing said host cells under conditions suitable for expression.

### BRIEF DESCRIPTION OF THE DRAWINGS

FIG. 1 represents the DNA sequence of BVH-NTHI1 gene from nontypeable H. influenzae strain 12085; SEQ ID NO: 1.

FIG. 2 represents the deduced amino acid sequence of the full-length BVH-NTHI1 from nontypeable H. influenzae strain 12085; SEQ ID NO: 2.

FIG. 3 represents the DNA sequence of BVH-NTHI2 gene from nontypeable H. influenzae strain 12085; SEQ ID NO: 3.

FIG. 4 represents the deduced amino acid sequence of the full-length BVH-NTHI2 from nontypeable H. influenzae strain 12085; SEQ ID NO: 4.

FIG. 5 represents the DNA sequence of BVH-NTHI3 gene from nontypeable H. influenzae strain 12085; SEQ ID NO: 5.

FIG. 6 represents the deduced amino acid sequence of the full-length BVH-NTHI3 from nontypeable H. influenzae strain 12085; SEQ ID NO: 6.

FIG. 7 represents the DNA sequence of BVH-NTHI4 gene from nontypeable H. influenzae strain 12085; SEQ ID NO: 7.

FIG. 8 represents the deduced amino acid sequence of the full-length BVH-NTHI4 from nontypeable H. influenzae strain 12085; SEQ ID NO: 8.

FIG. 9 represents the DNA sequence of BVH-NTHI5 gene from nontypeable H. influenzae strain 12085; SEQ ID NO: 9.

FIG. 10 represents the deduced amino acid sequence of the full-length BVH-NTHI5 from nontypeable H. influenzae strain 12085; SEQ ID NO: 10.

FIG. 11 represents the DNA sequence of BVH-NTHI6 gene from nontypeable H. influenzae strain 12085; SEQ ID NO: 11.

FIG. 12 represents the deduced amino acid sequence of the full-length BVH-NTHI6 from nontypeable H. influenzae strain 12085; SEQ ID NO: 12.

FIG. 13 represents the DNA sequence of BVH-NTHI7 gene from nontypeable H. influenzae strain 12085; SEQ ID NO: 13.

FIG. 14 represents the deduced amino acid sequence of the full-length BVH-NTHI7 from nontypeable H. influenzae strain 12085; SEQ ID NO: 14.

FIG. 15 represents the DNA sequence of BVH-NTHI8 gene from nontypeable H. influenzae strain 12085; SEQ ID NO: 15.

FIG. 16 represents the deduced amino acid sequence of the full-length BVH-NTHI8 from nontypeable H. influenzae strain 12085; SEQ ID NO: 16.

FIG. 17 represents the DNA sequence of BVH-NTHI9 gene from nontypeable H. influenzae strain 12085; SEQ ID NO: 17.

FIG. 18 represents the deduced amino acid sequence of the full-length BVH-NTHI9 from nontypeable H. influenzae strain 12085; SEQ ID NO: 18.

FIG. 19 represents the DNA sequence of BVH-NTHI10 gene from nontypeable H. influenzae strain 12085; SEQ ID NO: 19.

FIG. 20 represents the deduced amino acid sequence of the full-length BVH-NTHI10 from nontypeable H. influenzae strain 12085; SEQ ID NO: 20.

FIG. 21 represents the DNA sequence of BVH-NTHI11 gene from nontypeable H. influenzae strain 12085; SEQ ID NO: 21 FIG. 22 represents the deduced amino acid sequence of the full-length BVH-NTHI11 from nontypeable H. influenzae strain 12085; SEQ ID NO: 22.

FIG. 23 represents the DNA sequence of BVH-NTHI12 gene from nontypeable H. influenzae strain 12085; SEQ ID NO: 23

FIG. 24 represents the deduced amino acid sequence of the full-length BVH-NTHI12 from nontypeable H. influenzae strain 12085; SEQ ID NO: 24.

FIG. 25 depicts the comparison of the predicted amino acid sequences of the BVH-NTHI1 open reading frames from 12085, 10095, A18, and A108H. influenzae by using the program Clustal W from MacVector sequence analysis software (version 6.5). Underneath the alignment, there is a consensus line where (*) characters represent identical amino acid residues and (.) represent conserved amino acid residues.

### DETAILED DESCRIPTION OF THE INVENTION

The present invention provides purified and isolated polynucleotides, which encode H. influenzae polypeptides which may be used to prevent, treat, and/or diagnose H. influenzae infection. The present invention provides twelve separate preferred polynucleotides, each individually and separately defined by one SEQ ID NOs 1, 3, 5, 7, 9, 11, 13, 15, 17, 19, 21 and 23. Further provided in the present invention are twelve separate polypeptides, each individually and separately defined by one of seq ID NOS: 2, 4, 6, 8, 10, 12, 14, 16, 18, 20, 22 and 24. Those skilled in the art will appreciate that the invention includes polynucleotides that encode analogs such as mutants, variants, homologues and derivatives of such polypeptides, as described herein in the present patent application. The invention also includes RNA molecules corresponding to the DNA molecules of the invention. In addition to the DNA and RNA molecules, the invention includes the corresponding polypeptides and monospecific antibodies that specifically bind to such polypeptides.

According to one aspect, the present invention provides an isolated polynucleotide encoding a polypeptide having at least 70% identity to a second polypeptide comprising a sequence chosen from SEQ ID Nos: 2, 4, 6, 8, 10, 12, 14, 16, 18, 20, 22 and 24 or fragments or analogs thereof.

According to one aspect, the present invention provides an isolated polynucleotide encoding a polypeptide having at least 80% identity to a second polypeptide comprising a sequence chosen from SEQ ID Nos: 2, 4, 6, 8, 10, 12, 14, 16, 18, 20, 22 and 24 or fragments or analogs thereof.

According to one aspect, the present invention provides an isolated polynucleotide encoding a polypeptide having at least 85% identity to a second polypeptide comprising a sequence chosen from SEQ ID Nos: 2, 4, 6, 8, 10, 12, 14, 16, 18, 20, 22 and 24 or fragments or analogs thereof.

According to one aspect, the present invention provides an isolated polynucleotide encoding a polypeptide having at least 90% identity to a second polypeptide comprising a sequence chosen from SEQ ID Nos: 2, 4, 6, 8, 10, 12, 14, 16, 18, 20, 22 and 24 or fragments or analogs thereof.

According to one aspect, the present invention provides an isolated polynucleotide encoding a polypeptide having at least 95% identity to a second polypeptide comprising a sequence chosen from SEQ ID Nos: 2, 4, 6, 8, 10, 12, 14, 16, 18, 20, 22 and 24 or fragments or analogs thereof.

According to one aspect, the present invention provides an isolated polynucleotide encoding a polypeptide comprising a sequence chosen from: SEQ ID NOs: 2, 4, 6, 8, 10, 12, 14, 16, 18, 20, 22 and 24 or fragments or analogs thereof; According to one aspect, the present relates to polynucleotides encoding an epitope bearing portion of a polypeptide having a sequence chosen from SEQ ID NOs: 2, 4, 6, 8, 10, 12, 14, 16, 18, 20, 22 and 24 or fragments or analogs thereof.

According to one aspect, the present relates to polynucleotides encoding an epitope bearing portion of a polypeptide having a sequence chosen from SEQ ID NOs: 2, 4, 6, 8, 10, 12, 14, 16, 18, 20, 22 and 24.

According to one aspect, the present invention relates to epitope bearing portions of a polypeptide having a sequence chosen from SEQ ID NOs: 2, 4, 6, 8, 10, 12, 14, 16, 18, 20, 22 and 24 or fragments or analogs thereof.

According to one aspect, the present invention relates to epitope bearing portions of a polypeptide having a sequence chosen from SEQ ID NOs: 2, 4, 6, 8, 10, 12, 14, 16, 18, 20, 22 and 24.

According to one aspect, the present invention provides an isolated polynucleotide encoding a polypeptide having at least 70% identity to a second polypeptide comprising a sequence chosen from SEQ ID Nos: 2, 4, 6, 8, 10, 12, 14, 16, 18, 20, 22 and 24.

According to one aspect, the present invention provides an isolated polynucleotide encoding a polypeptide having at least 80% identity to a second polypeptide comprising a sequence chosen from SEQ ID Nos: 2, 4, 6, 8, 10, 12, 14, 16, 18, 20, 22 and 24.

According to one aspect, the present invention provides an isolated polynucleotide encoding a polypeptide having at least 85% identity to a second polypeptide comprising a sequence chosen from SEQ ID Nos: 2, 4, 6, 8, 10, 12, 14, 16, 18, 20, 22 and 24.

According to one aspect, the present invention provides an isolated polynucleotide encoding a polypeptide having at least 90% identity to a second polypeptide comprising a sequence chosen from SEQ ID Nos: 2, 4, 6, 8, 10, 12, 14, 16, 18, 20, 22 and 24.

According to one aspect, the present invention provides an isolated polynucleotide encoding a polypeptide having at least 95% identity to a second polypeptide comprising a sequence chosen from SEQ ID Nos: 2, 4, 6, 8, 10, 12, 14, 16, 18, 20, 22 and 24.

According to one aspect, the present invention provides an isolated polynucleotide encoding a polypeptide comprising a sequence chosen from: SEQ ID NOs: 2, 4, 6, 8, 10, 12, 14, 16, 18, 20, 22 and 24; According to one aspect, the present relates to polynucleotides encoding an epitope bearing portion of a polypeptide having a sequence chosen from SEQ ID NOs: 2, 41, 6, 8, 10, 12, 14, 16, 18, 20, 22 and 24.

In a further embodiment, the present invention also relates to polynucleotides encoding a polypeptide capable of generating antibodies having binding specificity for a polypeptide having a sequence chosen from SEQ ID NOs: 2, 4, 6, 8, 10, 12, 14, 16, 18, 20, 22 and 24 or fragments or analogs thereof.

In a further embodiment, the present invention also relates to polynucleotides encoding a polypeptide capable of generating antibodies having binding specificity for a polypeptide having a sequence chosen from SEQ ID NOs: 2, 4, 6, 8, 10, 12, 14, 16, 18, 20, 22 and 24.

The percentage of homology is defined as the sum of the percentage of identity plus the percentage of similarity or conservation of amino acid type.

One can use a program such as the CLUSTAL program to compare amino acid sequences. This program compares amino acid sequences and finds the optimal alignment by inserting spaces in either sequence as appropriate. It is possible to calculate amino acid identity or similarity (identity plus conservation of amino acid type) for an optimal alignment. A program like BLASTx will align the longest stretch of similar sequences and assign a value to the fit. It is thus possible to obtain a comparison where several regions of similarity are found, each having a different score. Both types of identity analysis are contemplated in the present invention.

In a further embodiment, the polypeptides in accordance with the present invention are antigenic.

In a further embodiment, the polypeptides in accordance with the present invention are immunogenic.

In a further embodiment, the polypeptides in accordance with the present invention can elicit an immune response in an individual.

In a further embodiment, the present invention also relates to polypeptides which are able to raise antibodies having binding specificity to the polypeptides of the present invention as defined above.

In a further embodiment, the present invention also relates to polypeptides capable of generating antibodies having binding specificity for a polypeptide having a sequence chosen from SEQ ID NOs: 2, 4, 6, 8, 10, 12, 14, 16, 18, 20, 22 and 24 or fragments or analogs thereof.

In a further embodiment, the present invention also relates to polypeptides capable of generating antibodies having binding specificity for a polypeptide having a sequence chosen from SEQ ID NOs: 2, 4, 6, 8, 10, 12, 14, 16, 18, 20, 22 and 24.

An antibody that “has binding specificity” is an antibody that recognises and binds the selected polypeptide but which does not substantially recognise and bind other molecules in a sample, e.g., a biological sample. Specific binding can be measured using an ELISA assay in which the selected polypeptide is used as an antigen.

In accordance with the present invention, “protection” in the biological studies is defined by a significant increase in the survival curve, rate or period. Statistical analysis using the Log rank test to compare survival curves, and Fisher exact test to compare survival rates and numbers of days to death, respectively, might be useful to calculate P values and determine whether the difference between the two groups is statistically significant. P values of 0.05 are regarded as not significant.

In an additional aspect of the invention there are provided antigenic/immunogenic fragments of the polypeptides of the invention, or of analogs thereof.

The fragments of the present invention should include one or more such epitopic regions or be sufficiently similar to such regions to retain their antigenic/immunogenic properties. Thus, for fragments according to the present invention the degree of identity is perhaps irrelevant, since they may be 100% identical to a particular part of a polypeptide or analog thereof as described herein. The present invention further provides fragments having at least 10 contiguous amino acid residues from the polypeptide sequences of the present invention. In one embodiment, at least 15 contiguous amino acid residues. In one embodiment, at least 20 contiguous amino acid residues.

The key issue, once again, is that the fragment retains the antigenic/immunogenic properties.

The skilled person will appreciate that fragments, analogs or derivatives of the polypeptides of the invention will also find use in the context of the present invention, i.e. as antigenic/immunogenic material. Thus, for instance polypeptides which include one or more additions, deletions, substitutions or the like are encompassed by the present invention.

As used herein, “fragments”, “analogs” or “derivatives” of the polypeptides of the invention include those polypeptides in which one or more of the amino acid residues are substituted with a conserved amino acid residue (preferably conserved) and which may be natural or unnatural. In one embodiment, derivatives and analogs of polypeptides of the invention will have about 70% identity with those sequences illustrated in the figures or fragments thereof. That is, 70% of the residues are the same. In a further embodiment, polypeptides will have greater than 80% identity. In a further embodiment, polypeptides will have greater than 90% identity. In a further embodiment, polypeptides will have greater than 95% identity. In a further embodiment, polypeptides will have greater than 99% identity. In a further embodiment, analogs of polypeptides of the invention will have fewer than about 20 amino acid residue substitutions, modifications or deletions and more preferably less than 10.

In one embodiment, derivatives and analogs of polypeptides of the invention will have about 70% homology with those sequences illustrated in the figures or fragments thereof. In a further embodiment, derivatives and analogs of polypeptides will have greater than 80% homology. In a further embodiment, derivatives and analogs of polypeptides will have greater than 90% homology. In a further embodiment, derivatives and analogs of polypeptides will have greater than 95% homology. In a further embodiment, derivatives and analogs of polypeptides will have greater than 99% homology. In a further embodiment, derivatives and analogs of derivatives and analogs of polypeptides of the invention will have fewer than about 20 amino acid residue substitutions, modifications or deletions and more preferably less than 10.

According to a further aspect, the invention provides polypeptides having at least 70% identity to a second polypeptide having an amino acid sequence chosen from: SEQ ID NOs: 0.2, 4, 6, 8, 10, 12, 14, 16, 18, 20, 22 and 24 or fragments or analogs thereof.

According to a further aspect, the invention provides polypeptides having at least 80% identity to a second polypeptide having an amino acid sequence chosen from: SEQ ID NOs: 2, 4, 6, 8, 10, 12, 14, 16, 18, 20, 22 and 24 or fragments or analogs thereof.

According to a further aspect, the invention provides polypeptides having at least 85% identity to a second polypeptide having an amino acid sequence chosen from: SEQ ID NOs: 2, 4, 6, 8, 10, 12, 14, 16, 18, 20, 22 and 24 or fragments or analogs thereof.

According to a further aspect, the invention provides polypeptides having at least 90% identity to a second polypeptide having an amino acid sequence chosen from: SEQ ID NOs: 2, 4, 6, 8, 10, 12, 14, 16, 18, 20, 22 and 24 or fragments or analogs thereof.

According to a further aspect, the invention provides polypeptides having at least 95% identity to a second polypeptide having an amino acid sequence chosen from: SEQ ID NOs: 2, 4, 6, 8, 10, 12, 14, 16, 18, 20, 22 and 24 or fragments or analogs thereof.

According to a further aspect, the invention provides polypeptides comprising a sequence chosen from: SEQ ID NOs: 2, 4, 6, 8, 10, 12, 14, 16, 18, 20, 22 and 24 or fragments or analogs thereof.

According to a further aspect, the invention provides polypeptides characterized by a sequence chosen from: SEQ ID NOs: 2, 4, 6, 8, 10, 12, 14, 16, 18, 20, 22 and 24 or fragments or analogs thereof.

According to a further aspect, the invention provides polypeptides having at least 70% identity to a second polypeptide having an amino acid sequence chosen from: SEQ ID NOs: 2, 4, 6, 8, 10, 12, 14, 16, 18, 20, 22 and 24.

According to a further aspect, the invention provides polypeptides having at least 80% identity to a second polypeptide having an amino acid sequence chosen from: SEQ ID NOs: 2, 4, 6, 8, 10, 12, 14, 16, 18, 20, 22 and 24.

According to a further aspect, the invention provides polypeptides having at least 85% identity to a second polypeptide having an amino acid sequence chosen from: SEQ ID NOs: 2, 4, 6, 8, 10, 12, 14, 16, 18, 20, 22 and 24.

According to a further aspect, the invention provides polypeptides having at least 90% identity to a second polypeptide having an amino acid sequence chosen from: SEQ ID NOs: 2, 4, 6, 8, 10, 12, 14, 16, 18, 20, 22 and 24.

According to a further aspect, the invention provides polypeptides having at least 95% identity to a second polypeptide having an amino acid sequence chosen from: SEQ ID NOs: 2, 4, 6, 8, 10, 12, 14, 16, 18, 20, 22 and 24.

According to a further aspect, the invention provides polypeptides comprising a sequence chosen from: SEQ ID NOs: 2, 4, 6, 8, 10, 12, 14, 16, 18, 20, 22 and 24.

According to a further aspect, the invention provides polypeptides characterized by a sequence chosen from: SEQ ID NOs: 2, 4, 6, 8, 10, 12, 14, 16, 18, 20, 22 and 24.

These substitutions are those having a minimal influence on the secondary structure and hydropathic nature of the polypeptide. Preferred substitutions are those known in the art as conserved, i.e. the substituted residues share physical or chemical properties such as hydrophobicity, size, charge or functional groups. These include substitutions such as those described by Dayhoff, M. in Atlas of Protein Sequence and Structure 5, 1978 and by Argos, P. in EMBO J. 8, 779-785, 1989. For example, amino acids, either natural or unnatural, belonging to one of the following groups represent conservative changes:

ala, pro, gly, gln, asn, ser, thr, val;

cys, ser, tyr, thr;

val, ile, leu, met, ala, phe;

lys, arg, orn, his;

and phe, tyr, trp, his.

The preferred substitutions also include substitutions of D-enantiomers for the corresponding L-amino acids.

Preferably, a fragment, analog or derivative of a polypeptide of the invention will comprise at least one antigenic region i.e. at least one epitope.

In an alternative approach, the analogs could be fusion polypeptides, incorporating moieties which render purification easier, for example by effectively tagging the desired polypeptide. It may be necessary to remove the “tag” or it may be the case that the fusion polypeptide itself retains sufficient antigenicity to be useful.

Thus, what is important for analogs, derivatives and fragments is that they possess at least a degree of the antigenicity/immunogenic of the protein or polypeptide from which they are derived.

Also included are polypeptides which have fused thereto other compounds which alter the biological or pharmacological properties of the polypeptide, i.e., polyethylene glycol (PEG) to increase half-life, leader or secretory amino acid sequences for ease of purification, prepro- and pro-sequences and (poly)saccharides.

Furthermore, in those situations where amino acid regions are found to be polymorphic, it may be desirable to vary one or more particular amino acids to more effectively mimic the different epitopes of the different H. influenzae strains.

Moreover, the polypeptides of the present invention can be modified by terminal —NH2 acylation (e.g. by acetylation or thioglycolic acid amidation, terminal carboxy amidation, e.g. with ammonia or methylamine) to provide stability, increased hydrophobicity for linking or binding to a support or other molecule.

Also contemplated are hetero and homo polypeptide multimers of the polypeptide fragments and analogues. These polymeric forms include, for example, one or more polypeptides that have been cross-linked with cross-linkers such as avidin/biotin, glutaraldehyde or dimethylsuperimidate. Such polymeric forms also include polypeptides containing two or more tandem or inverted contiguous sequences, produced from multicistronic mRNAs generated by recombinant DNA technology.

In a further embodiment, the present invention also relates to chimeric polypeptides which comprise one or more polypeptides or fragments or analogs or derivatives thereof as defined in the figures of the present application.

In a further embodiment, the present invention also relates to chimeric polypeptides comprising two or more polypeptides having a sequence chosen from SEQ ID NOs: 2, 4, 6, 8, 10, 12, 14, 16, 18, 20, 22 and 24 or fragments or analogs thereof; provided that the polypeptides are linked as to formed a chimeric polypeptide.

In a further embodiment, the present invention also relates to chimeric polypeptides comprising two or more polypeptides having a sequence chosen from SEQ ID NOs: 2, 4, 6, 8, 10, 12, 14, 16, 18, 20, 22 and 24; provided that the polypeptides are linked as to formed a chimeric polypeptide.

In order to achieve the formation of antigenic polymers (i.e. synthetic multimers), polypeptides may be utilized having bishaloacetyl groups, nitroarylhalides, or the like, where the reagents being specific for thio groups. Therefore, the link between two mercapto groups of the different peptides may be a single bound or may be composed of a linking group of at least two, typically at least four and not more than 16, but usually not more than about 14 carbon atoms.

In a particular embodiment, polypeptide fragments or analogs of the invention do not contain a methionine (Met) starting residue. Preferably, polypeptides will not incorporate a leader or secretory sequence (signal sequence). The signal portion of a polypeptide of the invention may be determined according to established molecular biological techniques. In general, the polypeptide of interest may be isolated from Haemophilus culture and subsequently sequenced to determine the initial residue of the mature protein and therefore the sequence of the mature polypeptide.

The following Table A describes the signal sequences of the polypeptides of the invention as described in the Figures.

| | SEQ | |
|---|---|---|
| | ID | |
| Polypeptide | No | Secretion signal |
| BVH-NTHI1 | 2 | MPVIRQVVFYDSLTGEQTKMKKFAGLITASFVA |
| BVH-NTHI2 | 4 | MKKLLKISAVSAALLSAPMMA |
| BVH-NTHI3 | 6 | MLMKLKATLTTLAAATLVLAA |
| BVH-NTHI4 | 8 | MMNRRHFIQIGATSILALSANRFAMA |
| BVH-NTHI5 | 10 | MKKIILTLSLGLLTAWSAQI |
| BVH-NTHI6 | 12 | no secretion signal |
| BVH-NTHI7 | 14 | MKKFLIAILLLILILAGAA |
| BVH-NTHI8 | 16 | MKMKKFILKSFLLATLGCVAFASMAQA |
| BVH-NTHI9 | 18 | MTMFKKISVLFFTLILAGCSSWS |
| BVH-NTHI10 | 20 | MSILLQGERFKKRLMPILLSMALAGCSNLLG |
| BVH-NTHI11 | 22 | MIRKLMKTPPFFTALFASAIFTLSVSQGVLG |
| BVH-NTHI12 | 24 | MKLKQLFAITAIA |

According to another aspect of the invention, there are also provided (i) a composition of matter containing a polypeptide of the invention, together with a carrier, diluent or adjuvant; (ii) a pharmaceutical composition comprising a polypeptide of the invention and a carrier, diluent or adjuvant; (iii) a vaccine comprising a polypeptide of the invention and a carrier, diluent or adjuvant; (iv) a method for inducing an immune response against Haemophilus, in an individual, by administering to the individual, an immunogenically effective amount of a polypeptide of the invention to elicit an immune response, e.g., a protective immune response to Haemophilus; and particularly, (v) a method for preventing and/or treating a Haemophilus infection, by administering a prophylactic or therapeutic amount of a polypeptide of the invention to an individual in need.

Before immunization, the polypeptides of the invention can also be coupled or conjugated to carrier proteins such as tetanus toxin, diphtheria toxin, hepatitis B virus surface antigen, poliomyelitis virus VP1 antigen or any other viral or bacterial toxin or antigen or any suitable proteins to stimulate the development of a stronger immune response. This coupling or conjugation can be done chemically or genetically. A more detailed description of peptide-carrier conjugation is available in Van Regenmortel, M. H. V., Briand J. P., Muller S., Plaue S., <<Synthetic Polypeptides as antigens>> in Laboratory Techniques in Biochemistry and Molecular Biology, Vol.19 (ed.) Burdou, R. H. & Van Knippenberg P. H. (1988), Elsevier New York.

According to another aspect, there are also provided pharmaceutical compositions comprising one or more Haemophilus polypeptides of the invention in a mixture with a pharmaceutically acceptable carrier, diluent or adjuvant. Suitable adjuvants include (1) oil-in-water emulsion formulations such as MF59′, SAF™, Ribi™; (2) Freund's complete or incomplete adjuvant; (3) salts i.e. AlK(SO₄)₂, AlNa(SO₄)₂, AlNH₄(SO₄)₂, Al(OH)₃, AlPO₄, silica, kaolin; (4) saponin derivatives such as Stimulon™ or particles generated therefrom such as ISCOMs (immunostimulating complexes); (5) cytokines such as interleukins, interferons, macrophage colony stimulating factor (M-CSF), tumor necrosis factor (TNF); (6) other substances such as carbon polynucleotides i.e. poly IC and poly AU, detoxified cholera toxin (CTB) and E. coli heat labile toxin for induction of mucosal immunity. A more detailed description of adjuvant is available in a review by M. Z. I Khan et al. in Pharmaceutical Research, vol. 11, No. 1 (1994) pp2-11, and also in another review by Gupta et al., in Vaccine, Vol. 13, No. 14, pp1263-1276 (1995) and in WO 99/24578. Preferred adjuvants include QuilA™, QS21™, Alhydrogel™ and Adjuphos™.

Pharmaceutical compositions of the invention may be administered parenterally by injection, rapid infusion, nasopharyngeal absorption, dermoabsorption, or buccal or oral.

Pharmaceutical compositions of the invention are used for the treatment or prophylaxis of Haemophilus infection and/or diseases and symptoms mediated by Haemophilus infection as described in P. R. Murray (Ed, in chief), E. J. Baron, M. A. Pfaller, F. C. Tenover and R. H. Yolken. Manual of Clinical Microbiology, ASM Press, Washington, D. C. seventh edition, 1999, p1481-1482 which are herein incorporated by reference. In one embodiment, pharmaceutical compositions of the present invention are used for the treatment or prophylaxis of otitis media (acute or recurrent), sinusitis, bronchitis, pneumonia, meningitis and bacteremia. In one embodiment, pharmaceutical compositions of the invention are used for the treatment or prophylaxis of Haemophilus infection and/or diseases and symptoms mediated by Haemophilus infection. In a further embodiment, the Haemophilus infection is Haemophilus Inf luenzae. In a further embodiment, the Haemophilus infection is Nontypeable Haemophilus Influenzae. In a further embodiment, the Haemophilus infection is Typeable Haemophilus Influenzae.

As used in the present application, the term “individual” include mammal. In a further embodiment, the mammal is human.

In a particular embodiment, pharmaceutical compositions are administered to those individuals at risk of H. influenzae infection such as infants, elderly and immunocompromised individuals.

Pharmaceutical compositions are preferably in unit dosage form of about 0.001 to 100 μg/kg (antigen/body weight) and more preferably 0.01 to 10 μg/kg and most preferably 0.1 to 1 μg/kg 1 to 3 times with an interval of about 1 to 6 week intervals between immunizations.

Pharmaceutical compositions are preferably in unit dosage form of about 0.1 μg to 10 mg and more preferably 1 μg to 1 mg and most preferably 10 to 100 μg 1 to 0.3 times with an interval of about 1 to 6 week intervals between immunizations.

In one embodiment, polynucleotides are those illustrated in SEQ ID Nos: 1, 3, 5, 7, 9, 11, 13, 15, 17, 19, 21 and 23 which may include the open reading frames (ORF), encoding the polypeptides of the invention.

In a further embodiment, polynucleotides are those illustrated in SEQ ID NO: 1, 3, 5, 7, 9, 11, 13, 15, 17, 19, 21, 23 encoding polypeptides of the invention.

It will be appreciated that the polynucleotide sequences illustrated in the figures may be altered with degenerated codons yet still encode the polypeptide of the invention. Accordingly the present invention further provides polynucleotide herein above described (or the complement sequence thereof) having 50% identity between sequences. In one embodiment, at least 70% identity between sequences. In one embodiment, at least 75% identity between sequences. In one embodiment, at least 80% identity between sequences. In one embodiment, at least 85% identity between sequences. In one embodiment, at least 90% identity between sequences. In a further embodiment, polynucleotides are hybridizable under stringent conditions, i.e. having at least 95% identity. In a further embodiment, more than 97% identity.

Suitable stringent conditions for hybridation can be readily determined by one of skilled in the art (see for example Sambrook et al., (1989) Molecular cloning: A Laboratory Manual, 2 ed, Cold Spring Harbor, N.Y.; Current Protocols in Molecular Biology, (1999) Edited by Ausubel F. M. et al., John Wiley & Sons, Inc., N.Y.).

In a further embodiment, the present invention provides polynucleotides that hybridize under stringent conditions to either

(a) a DNA sequence encoding a polypeptide or

(b) the complement of a DNA sequence encoding a polypeptide;

wherein said polypeptide comprises SEQ ID NO: 2, 4, 6, 8, 10, 12, 14, 16, 18, 20, 22, 24, or fragments or analogs thereof.

In a further embodiment, the present invention provides polynucleotides that hybridize under stringent conditions to either

(a) a DNA sequence encoding a polypeptide or

(b) the complement of a DNA sequence encoding a polypeptide;

wherein said polypeptide comprises at least 10 contiguous amino acid residues from a polypeptide comprising SEQ ID NO: 2, 4, 6, 8, 10, 12, 14, 16, 18, 20, 22, 24 or fragments or analogs thereof.

In a further embodiment, the present invention provides polynucleotides that hybridize under stringent conditions to either

(a) a DNA sequence encoding a polypeptide or

(b) the complement of a DNA sequence encoding a polypeptide;

wherein said polypeptide comprises SEQ ID NO: 2, 4, 6, 8, 10, 12, 14, 16, 18, 20, 22, 24.

In a further embodiment, the present invention provides polynucleotides that hybridize under stringent conditions to either

(a) a DNA sequence encoding a polypeptide or

(b) the complement of a DNA sequence encoding a polypeptide;

wherein said polypeptide comprises at least 10 contiguous amino acid residues from a polypeptide comprising SEQ ID NO: 2, 4, 6, 8, 10, 12, 14, 16, 18, 20, 22, 24.

As will be readily appreciated by one skilled in the art, polynucleotides include both DNA and RNA.

The present invention also includes polynucleotides complementary to the polynucleotides described in the present application.

In a further aspect, polynucleotides encoding polypeptides of the invention, or fragments or analogs thereof, may be used in a DNA immunization method. That is, they can be incorporated into a vector which is replicable and expressible upon injection thereby producing the antigenic polypeptide in vivo. For example polynucleotides may be incorporated into a plasmid vector under the control of the CMV promoter which is functional in eukaryotic cells. Preferably, the vector is injected intramuscularly.

According to another aspect, there is provided a process or method of manufacturing for producing polypeptides of the invention by recombinant techniques by expressing a polynucleotide encoding said polypeptide in a host cell and recovering the expressed polypeptide product. Alternatively, the polypeptides can be produced according to established synthetic chemical techniques, i.e. solution phase or solid phase synthesis of oligopeptides which are ligated to produce the full polypeptide (block ligation).

General methods for obtention and evaluation of polynucleotides and polypeptides are described in the following references: Sambrook et al., (1989) Molecular cloning: A Laboratory Manual, 2^{nd} ed, Cold Spring Harbor, N.Y.; Current Protocols in Molecular Biology, (1999) Edited by Ausubel F. M. et al., John Wiley & Sons, Inc., N.Y.; PCR Cloning Protocols, from Molecular Cloning to Genetic Engineering, (1997) Edited by White B. A., Humana Press, Totowa, N.J., 490 pages; Protein Purification, Principles and Practices, (1993) Scopes R. K., Springer-Verlag, N.Y., 3^{rd} Edition, 380 pages; Current Protocols in Immunology, (1999) Edited by Coligan J. E. et al., John Wiley & Sons Inc., N.Y., are herein incorporated by reference.

For recombinant production, host cells are transfected with vectors which encode the polypeptides of the invention, and then cultured in a nutrient media modified as appropriate for activating promoters, selecting transformants or amplifying the genes. Suitable vectors are those that are viable and replicable in the chosen host and include chromosomal, non-chromosomal and synthetic DNA sequences e.g. bacterial plasmids, phage DNA, baculovirus, yeast plasmids, vectors derived from combinations of plasmids and phage DNA. The polypeptide sequence may be incorporated in the vector at the appropriate site using restriction enzymes such that it is operably linked to an expression control region comprising a promoter, ribosome binding site (consensus region or Shine-Dalgarno sequence), and optionally an operator (control element). One can select individual components of the expression control region that are appropriate for a given host and vector according to established molecular biology principles (Sambrook et al., (1989) Molecular Cloning: A Laboratory manual, 2^{nd} ed., Cold Spring Harbor, N.Y.; Current Protocols in Molecular Biology, (1999) Edited by Ausubel F. M. et al., John Wikey & Sons, Inc., N.Y., incorporated herein by reference). Suitable promoters include but are not limited to LTR or SV40 promoter, E. coli lac, tac or trp promoters and the phage lambda P_{L} promoter. Vectors will preferably incorporate an origin of replication as well as selection markers i.e. ampicilin resistance gene. Suitable bacterial vectors include pET, pQE70, pQE60, pQE-9, pD10 phagescript, psiX174, pbluescript SK, pbsks, pNH8A, pNH16a, pNH18A, pNH46A, ptrc99a, pKK223-3, pKK233-3, pDR540, PRITS and eukaryotic vectors pBlueBacIII, pWLNEO, pSV2CAT, pOG44, pXT1, pSG, pSVK3, pBPV, pMSG and pSVL. Host cells may be bacterial i.e. E. coli, Bacillus subtilis, Streptomyces; fungal i.e. Aspergillus niger, Aspergillus nidulins; yeast i.e. Saccharomyces or eukaryotic i.e. CHO, COS.

Upon expression of the polypeptide in culture, cells are typically harvested by centrifugation then disrupted by physical or chemical means (if the expressed polypeptide is not secreted into the media) and the resulting crude extract retained to isolate the polypeptide of interest. Purification of the polypeptide from culture media or lysate may be achieved by established techniques depending on the properties of the polypeptide i.e. using ammonium sulfate or ethanol precipitation, acid extraction, anion or cation exchange chromatography, phosphocellulose chromatography hydrophobic interaction chromatography, hydroxylapatite chromatography and lectin chromatography. Final purification may be achieved using HPLC.

The polypeptide may be expressed with or without a leader or secretion sequence. In the former case, the leader may be removed using post-translational processing (see U.S. Pat. No. 4,431,739, U.S. Pat. No. 4,425,437 and U.S. Pat. No. 4,338,397 incorporated herein by reference) or be chemically removed subsequent to purifying the expressed polypeptide.

According to a further aspect, the Haemophilus polypeptides of the invention may be used in a diagnostic test for H. influenzae infection, in particular for H. influenzae infection. Several diagnostic methods are possible, for example detecting Haemophilus organismn in a biological sample, the following procedure may be followed:

a. obtaining a biological sample from an individual;

b. incubating an antibody or fragment thereof reactive with an Haemophilus polypeptide of the invention with the biological sample to form a mixture, and

c. detecting specifically bound antibody or bound fragment in the mixture which indicates the presence of Haemophilus.

Alternatively, a method for the detection of antibody specific to an H. influenzae antigen in a biological sample containing or suspected of containing said antibody may be performed as follows:

a. obtaining a biological sample from an individual;

b. incubating one or more H. influenzae polypeptides of the invention or fragments thereof with the biological sample to form a mixture; and

c. detecting specifically bound antigen or bound fragment in the mixture which indicates the presence of antibody specific to H. influenzae.

One of skill in the art will recognize that this diagnostic test may take several forms, including an immunological test such as an enzyme-linked immunoadsorbent assay (ELISA), a radioimmunoassay or a latex agglutination assay, essentially to determine whether antibodies specific for the polypeptides are present in an organism.

According to a further aspect, the invention relates to a method for prophylactic or therapeutic treatment of otitis media, sinusitis, bronchitis, pneumonia and meningitis and bacteremia comprising administering to an individual a therapeutic or prophylactic amount of a composition of the invention.

According to a further aspect, the invention relates to a method for prophylactic or therapeutic treatment of Haemophilus influenzae bacterial infection in an individual susceptible to Haemophilus influenzae infection comprising administering to said individual a therapeutic or prophylactic amount of a composition of the invention. In a further embodiment, Haemophilus influenzae is Nontypeable Haemophilus influenzae. In a further embodiment, Haemophilus influenzae is Typeable Haemophilus influenzae.

According to a further aspect, the invention relates to a method for diagnostic of otitis media, sinusitis, bronchitis, pneumonia and meningitis and bacteremia comprising administering to an individual a therapeutic or prophylactic amount of a composition of the invention.

According to a further aspect, the invention relates to a method for diagnostic of Haemophilus influenzae bacterial infection in an individual susceptible to Haemophilus influenzae infection comprising administering to said individual a therapeutic or prophylactic amount of a composition of the invention. In a further embodiment, Haemophilus influenzae is Nontypeable Haemophilus influenzae. In a further embodiment, Haemophilus influenzae is Typeable Haemophilus influenzae.

According to a further aspect, the invention relates to the use of a pharmaceutical composition of the invention for the prophylactic or therapeutic treatment of Haemophilus infection comprising administering to said individual a prophylactic or therapeutic amount of the composition.

According to a further aspect, the Haemophilus polypeptides of the invention may be used in a kit comprising the polypeptides of the invention for detection of diagnosis of Hameophilus infection.

The DNA sequences encoding polypeptides of the invention may also be used to design DNA probes for use in detecting the presence of H. influenzae in a biological sample suspected of containing such bacteria. The detection method of this invention comprises:

a. obtaining the biological sample from an individual;

b. incubating one or more DNA probes having a DNA sequence encoding a polypeptide of the invention or fragments thereof with the biological sample to form a mixture; and

c. detecting specifically bound DNA probe in the mixture which indicates the presence of H. influenzae bacteria.

The DNA probes of this invention may also be used for detecting circulating H. influenzae (i.e. H. influenzae nucleic acids) in a sample, for example using a polymerase chain reaction, as a method of diagnosing H. influenzae infections. The probe may be synthesized using conventional techniques and may be immobilized on a solid phase or may be labelled with a detectable label. A preferred DNA probe for this application is an oligomer having a sequence complementary to at least about 6 contiguous nucleotides of the H. influenzae polypeptides of the invention.

Another diagnostic method for the detection of H. influenzae in an individual comprises

a. labelling an antibody reactive with a polypeptide of the invention or fragment thereof with a detectable label;

b. administering the labelled antibody or labelled fragment to the individual; and

c. detecting specifically bound labelled antibody or labelled fragment in the individual which indicates the presence of H. influenzae.

A further aspect of the invention is the use of the Haemophilus polypeptides of the invention as immunogens for the production of specific antibodies for the diagnosis and in particular the treatment of H. influenzae infection. Suitable antibodies may be determined using appropriate screening methods, for example by measuring the ability of a particular antibody to passively protect against H. influenzae infection in a test model. One example of an animal model is the mouse model described in the example herein. The antibody may be a whole antibody or an antigen-binding fragment thereof and may belong to any immunoglobulin class. The antibody or fragment may be of animal origin, specifically of mammalian origin and more specifically of murine, rat or human origin. It may be a natural antibody or a fragment thereof, or if desired, a recombinant antibody or antibody fragment. The term recombinant antibody or antibody fragment means antibody or antibody fragment which was produced using molecular biology techniques. The antibody or antibody fragments may be polyclonal or preferably monoclonal. It may be specific for a number of epitopes associated with the H. influenzae polypeptides but is preferably specific for one.

A further aspect of the invention is the use of a pharmaceutical composition of the invention for the prophylactic or therapeutic treatment of Haemophilus infection comprising administering to said individual a prophylactic or therapeutic amount of the composition.

Unless otherwise defined, all technical and scientific terms used herein have the same meaning as commonly understood by one of ordinary skill in the art to which this invention belong. All publications, patent applications, patents and other references mentioned herein are incorporated by reference in their entirety. In case of conflict, the present specification including definitions, will control. In addition, the materials, methods, and examples are illustrative only and not intended to be limiting.

Isolation of Nontypeable H. influenzae (NTHI) Polypeptides and Identification of Genes Encoding these Polypeptides

Isolation of Surface-Exposed NTHI Polypeptides

Bacterial Strains

Nontypeable H. influenzae clinical isolates 12085 and 10095 were provided by D. M. Granoff (St-Louis, Mo.) and B-20, A18, A108, C-98, C-26 and B-31 by the Centre de Recherche en Infectiologie du Centre Hospitalier de l'Universite Laval.

Nontypeable H. influenzae 12085 was isolated from the blood of a child with lower respiratory tract infection in Pakistan. Escherichia coli DH5α (GIBCO BRL, Burlington, Ontario) was used as the host strain for recombinant DNA. E. coli XL1-Blue MRF′ (Stratagene, LaJolla, Calif.) was used as the host strain for infection with lambda ZAP Express phage vector. E. coli XLOLR (Stratagene) was used as the host strain for infection with in vivo excised filamentous lambda ZAP Express.

Antisera

Mice antisera was produced following three-week intervals subcutaneous immunization with outer membrane proteins in presence of complete or incomplete Freund adjuvants (Cedarlane Laboratories Ltd, Hornby, Canada). Sera was collected 21 days after the tertiary immunization.

Four human antisera were selected on the basis of their reactivity by ELISA and Western blotting on heat-killed nontypeable H. influenzae 12085.

H. influenzae Library Construction and Screening

Nontypeable H. influenzae 12085 DNA was extracted using a genomic extraction Kit (QIAgen). DNA was partially digested with Tsp509I and ligated to EcoRI digested λ-ZAP Express phage arms (Stratagene). The ligation was packaged in vitro with Gigapack extracts according to the manufacturer's recommendations (Stratagene). Recombinant phage were plated on E. coli XL1-Blue MRF′ at density of 2.5×10⁴ PFU/150 mm (diameter) plate. Following eight hours of incubation, the plates were overlaid with nitrocellulose disks and the resulting lifts were processed for immunoblotting with human and mice sera. Positive clones were picked up and plaque-purified. Recombinant pBK-CMV plasmids coding for the Haemophilus genes of interest were recovered from the purified bacteriophage using ExAssist filamentous helper phage and the lambda-resistant strain E. coli XLOLR by in vivo excision.

Identification of Antigens

SDS-PAGE and immunoblot analysis of lysates of these recombinant E. coli showed that each clone expressed one or more polypeptides that reacted with human or mice antisera.

DNA Sequencing and Sequence Analysis

The genomic clones were sequenced with the Taq DyeDeoxy Terminator Cycle Sequencing Kit (Applied Biosystem, Foster City, Calif.). Several internal primers were designed to sequence further into the cloned inserts. Sequence assembly was performed using Sequencher software (Gene Codes Corporation, Ann Arbor, Mich.) and sequence analysis was performed with McVector software (Oxford Molecular Ltd., Campbell, Calif.).

### EXAMPLE 1

This example illustrates the cloning of nontypeable H. influenzae genes into an expression vector

The coding regions of nontypeable H. influenzae genes BVH-NTHI1 to BVH-NTHI12 (SEQ ID NO:1,3,5,7,9,11,13,15,17,19,21 and 23) were amplified by PCR (DNA Thermal Cycler GeneAmp PCR system 2400 Perkin Elmer, San Jose, Calif.) from genomic DNA of nontypeable H. influenzae strain 12085 by using the following oligonucleotide primers that contained base extensions for the addition of restriction sites NcoI (CCATGG), NdeI (CATATG), SalI (GTCGAC) or XhoI (CTCGAG) (Table 1). PCR products were purified from agarose gels by using a QIAquick gel extraction kit from QIAgen following the manufacturer's instructions (Chatsworth, Calif.), and digested with appropriate restriction endonucleases (Pharmacia Canada Inc, Baie d'Urfe, Canada). The pET vectors (Novagen, Madison, Wis.) were digested likewise and purified from agarose gels using a QIAquick gel extraction kit from QIAgen (Chatsworth, Calif.). The digested PCR products were ligated to the enzyme-restricted pET expression vector. The ligated products were transformed into E. coli strain DH5α [φ80dlacZΔM15 Δ(lacZYA-argF)U169 endA1 recAl1 hsdR17(r_{K}−m_{K}+) deoR thi-1 supE44 λ⁻gyrA96 relA1] (Gibco BRL, Gaithersburg, Md.) according to the method of Simanis (Hanahan D. (1985) In: DNA Cloning. D. M. Glover, ed., IRL Press, Washington D. C. vol. 1 pp. 109-135). Recombinant gene-containing plasmids (rpET) were purified using a QIAgen kit (Chatsworth, Calif.) and DNA inserts were sequenced (Taq Dye Deoxy Terminator Cycle Sequencing kit, ABI, Foster City, Calif.).

TABLE 1
List of oligonucleotide primers for DNA cloning.

| PCR | | | | Restric- | Cloning |
|---|---|---|---|---|---|
| Primer | Seq. | | Nucleotide | tion | vector- |
| set | ID. | Sequence 5′-3′ | position* | sites | Strain |
| HAMJ342- | 25 | CAAGGCGTTTTCATATG | 1-1137 | NdeI | pET21-b + |
| | | CCTGTCATTCGGCAGG | | | Tuner (DE3) |
| HAMJ343 | 26 | CTAATTGACCTCGAGTT | | XhoI | |
| | | TTGCTGCTTTTAATTCT | | | |
| | | TGATAATATTG | | | |
| HAMJ345- | 27 | GTAAGGAAACATACATA | 1-1005 | NdeI | pET21-b + |
| | | TGAAAAAACTTTTAAAA | | | BL21 (DE3) |
| | | ATTAGTG | | | |
| HAMJ344 | 28 | TTAGAGACTCGAGTTTA | | XhoI | |
| | | GCTAAACATTCTATGTA | | | |
| | | GCTATC | | | |
| HAM4J348- | 29 | CCTAACATTGACATATG | 1-1647 | NdeI | pET21-b + |
| | | CTTATGAAACTAAAAGC | | | AD494 (DE3) |
| | | AACA | | | |
| HAMJ349 | 30 | TCAATATCTCGAGTTTA | | XhoI | |
| | | CCATCAACACTCACACC | | | |
| HAMJ346- | 31 | CTAATAAGGAAAACCAT | 1-1971 | NdeI | pET21-b + |
| | | ATGATGAACAGACGTCA | | | Tuner (DE3) |
| | | TTTTATTC | | | |
| HAMJ399 | 32 | GACCTAGCTCGAGTTTA | | XhoI | |
| | | GATAAATCAATTTGATA | | | |
| | | CACTG | | | |
| HAMJ376- | 33 | CATAAGGAGTAACATAT | 1-480 | NdeI | pET21-b + |
| | | GAAAAAAATTATTTTAA | | | Tuner (DE3) |
| | | CATTATC | | | |
| | | GTGCAGATTCTCGAGTT | | XhoI | |
| HAMJ377 | 34 | TTTTATCAACTGAA | | | |
| HAMJ460- | 35 | CTGGACAGACCATATGC | 2-936 | NdeI | pET21-b + |
| | | CTTCTGATTTAGTCGC | | | Tuner (DE3) |
| HAMJ461 | 36 | CTAAATTGAGCTCGAGA | | XhoI | |
| | | TTAGTTTTTAATTTACT | | | |
| | | CC | | | |
| HAMJ458- | 37 | CTTTCTTATAGCATATG | 1-1041 | NdeI | pET21-b + |
| | | AAGAAATTTTTAATTGC | | | Tuner (DE |
| | | GATT | | | 3)pLysS |
| HAMJ459 | 38 | CTTCCGCACTCGAGTTT | | XhoI | |
| | | GGCATTTTTC | | | |
| HAMJ477- | 39 | GGAAGGAGCTAGCATGA | 1-939 | NheI | pET21-b + |
| | | AAATGAAAAAATTTATT | | | Tuner (DE3) |
| | | C | | | |
| HAMJ478 | 40 | TTGATACTCTCGAGTTT | | XhoI | |
| | | ATTAAAATACTG | | | |
| HAMJ481- | 41 | CTTTAATCACATATGAC | 1-534 | NdeI | pET21-b + |
| | | TATGTTTAAAAAAATCT | | | |
| | | CTG | | | |
| HAMJ482 | 42 | CACCAATCTCGAGTTTA | | XhoI | |
| | | GATGTACAGGCTT | | | |
| HAMJ78- | 43 | ACCCGCATGGCTGTTCA | 75-1728 | Nco1 | pET32-c + |
| | | AATCTACTTGGTAG | | | AD494 (DE3) |
| HAMJ79 | 44 | ACTCGTCGACTTAGTTT | | | |
| | | GCAACTGGTACAAT | | SalI | |
| HAMJ414- | 45 | GATTAGGGAAAACATAT | 1-3336 | NdeI | pET21-b + |
| | | GATTAGGAAACTTATGA | | | |
| | | A | | | |
| HAMJ415 | 46 | CCATAATTTGCTCGAGT | | XhoI | |
| | | TTTTTTACATCAAC | | | |
| HAMJ450- | 47 | GGAAGGAGCTAGCATGA | 1-816 | NheI | pET21-b + |
| | | AATTAAAACAACTTTTT | | | Tuner (DE3) |
| | | G | | | |
| HAMJ411 | 48 | GTGCGGTAGCTCGAGCC | | XhoI | |
| | | AACCTTTTAC | | | |

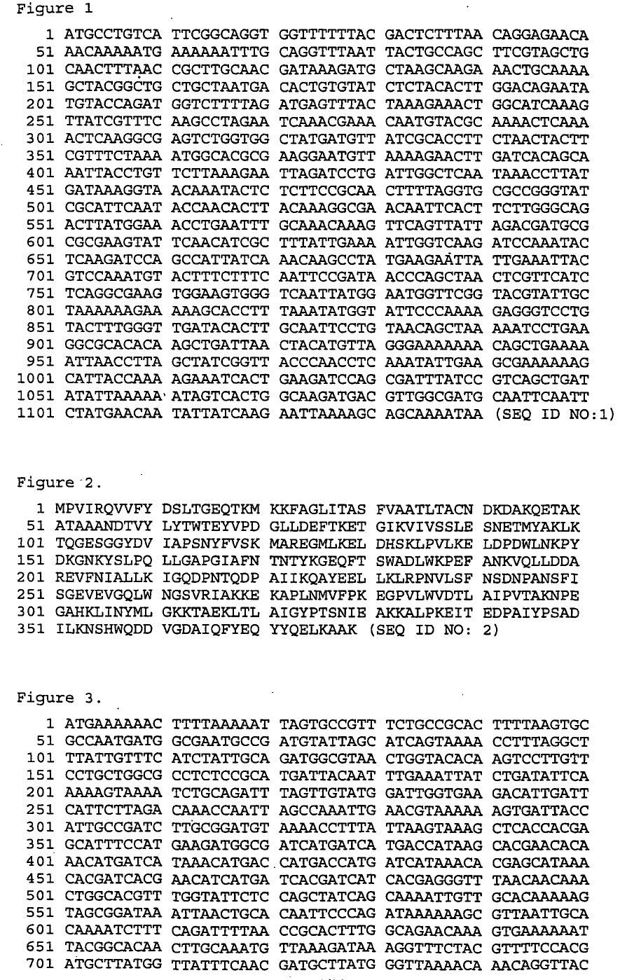

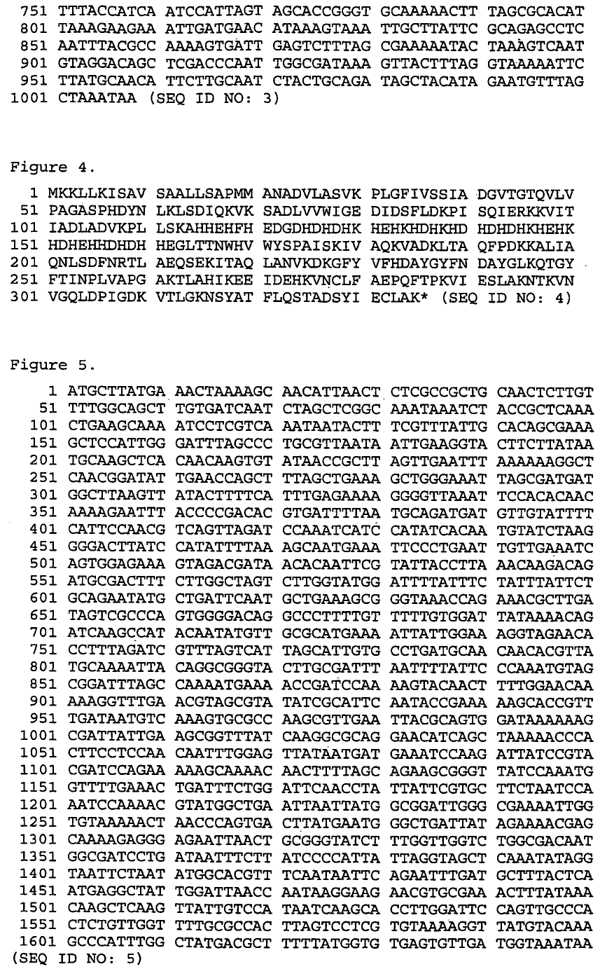

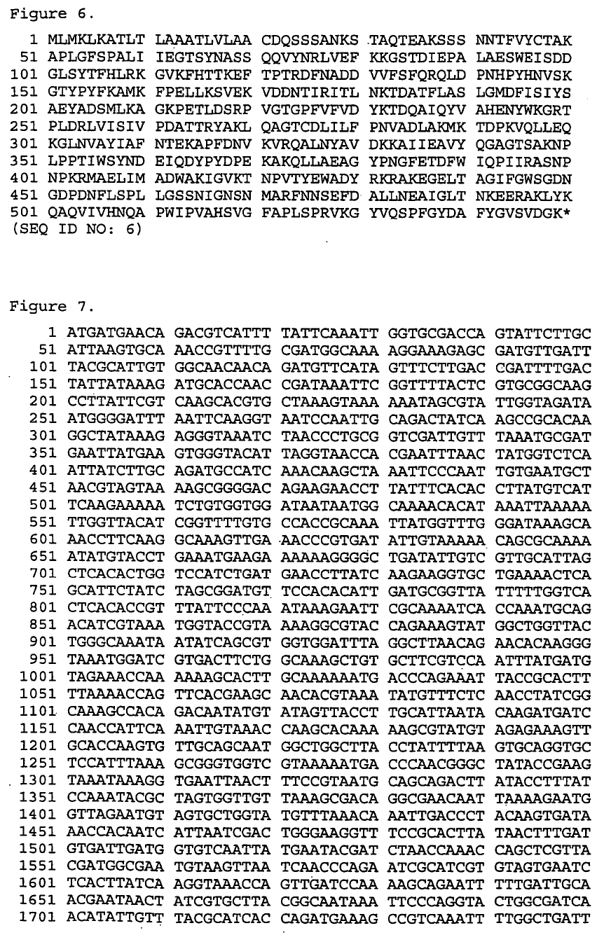

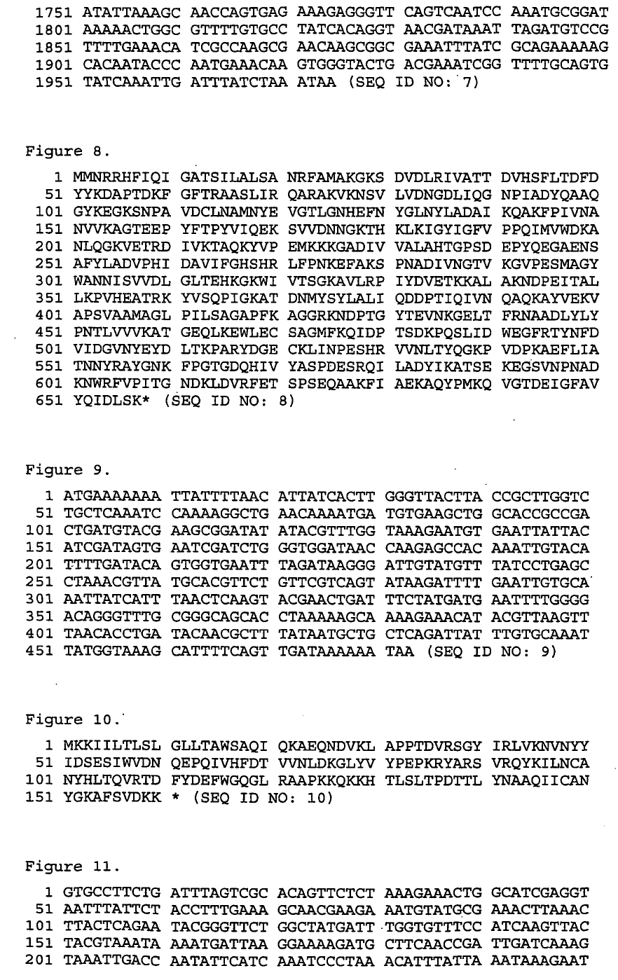

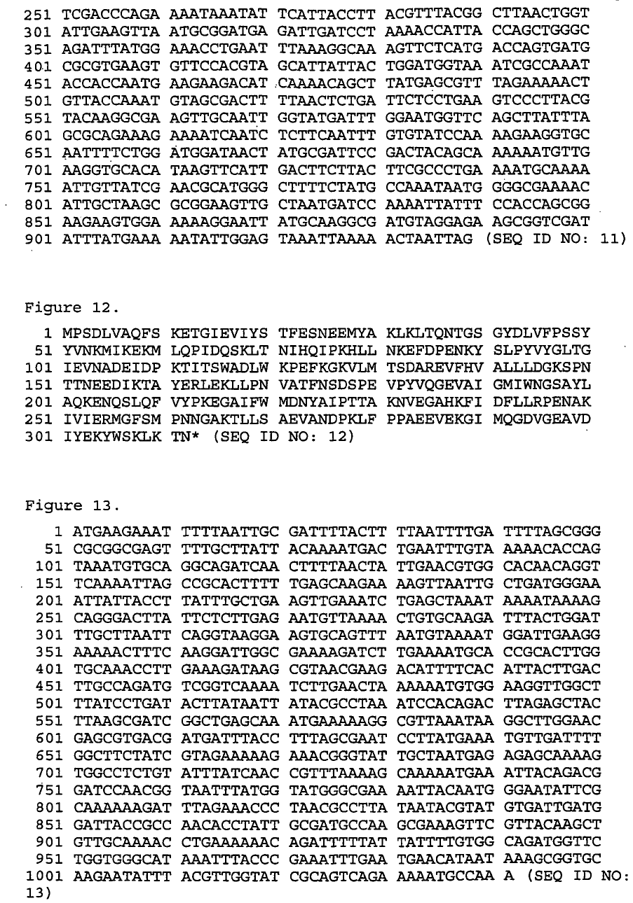

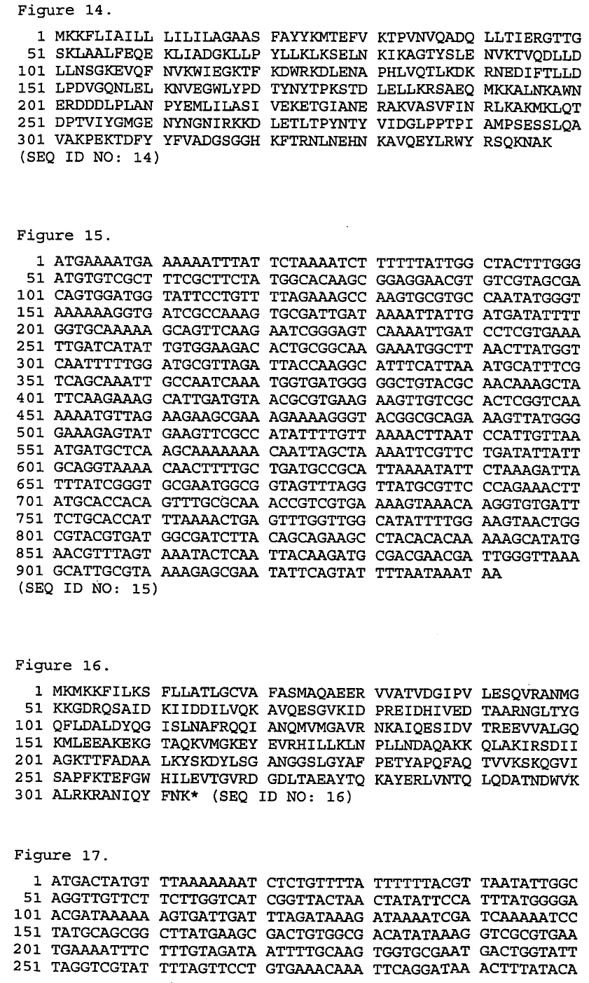

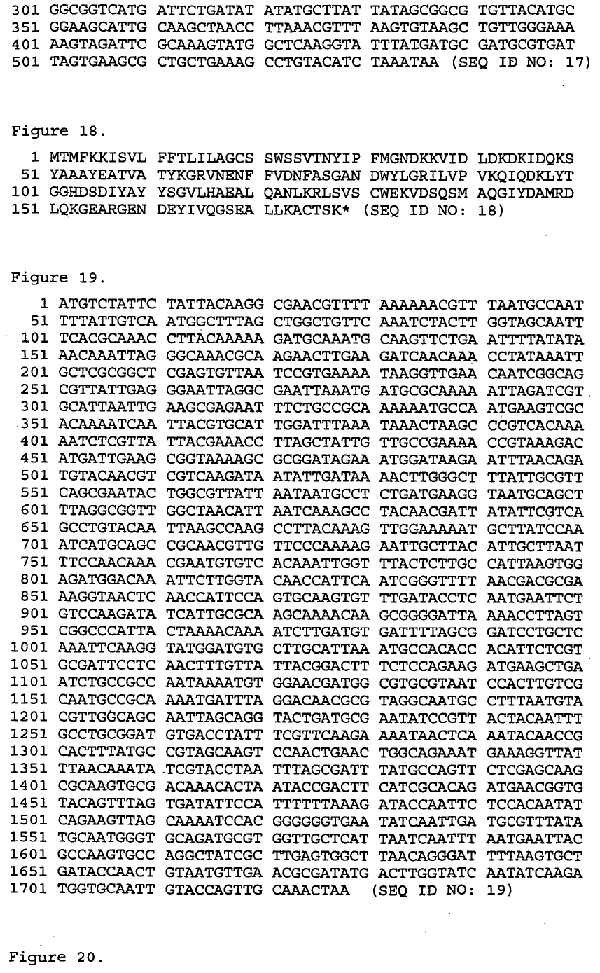

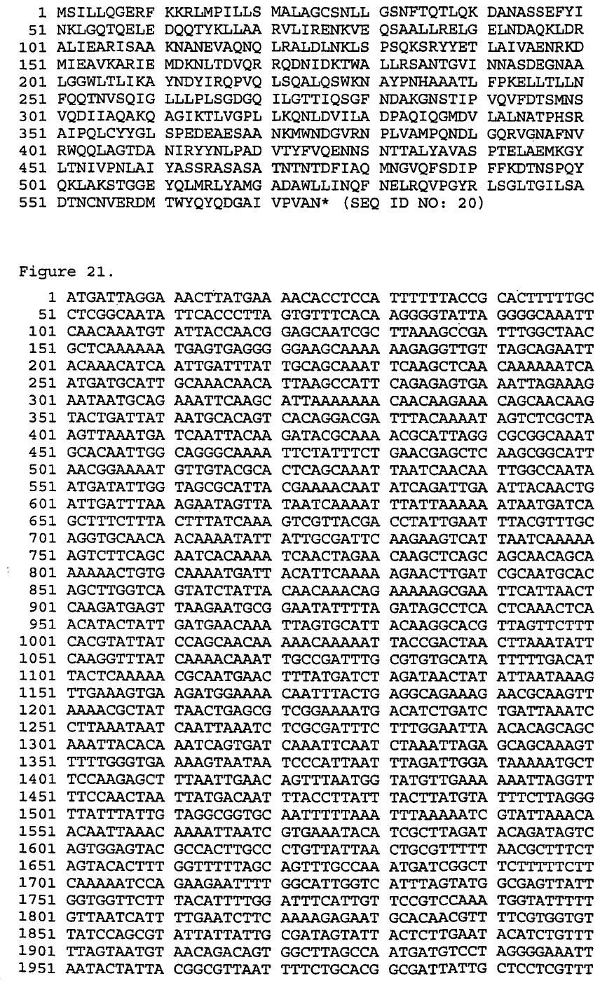

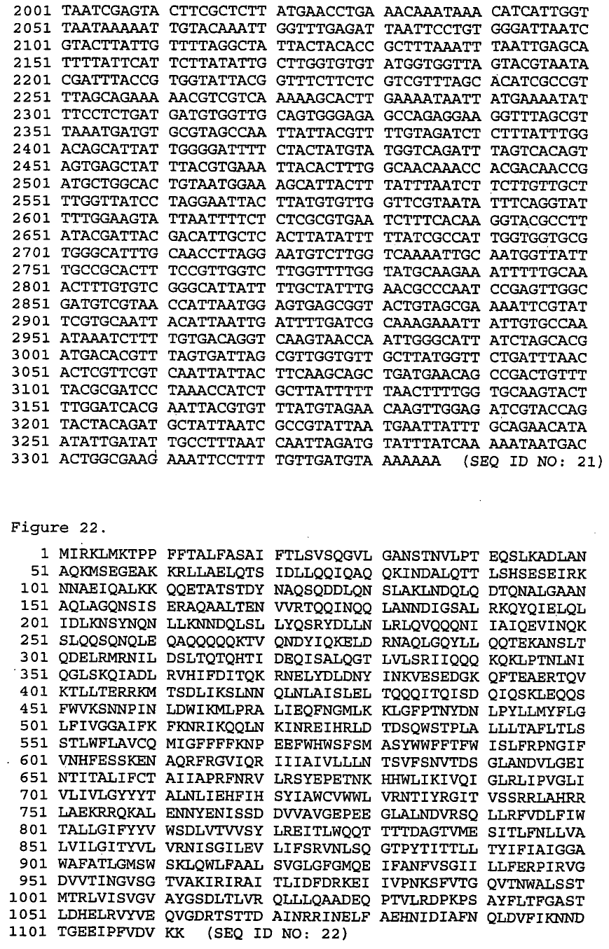

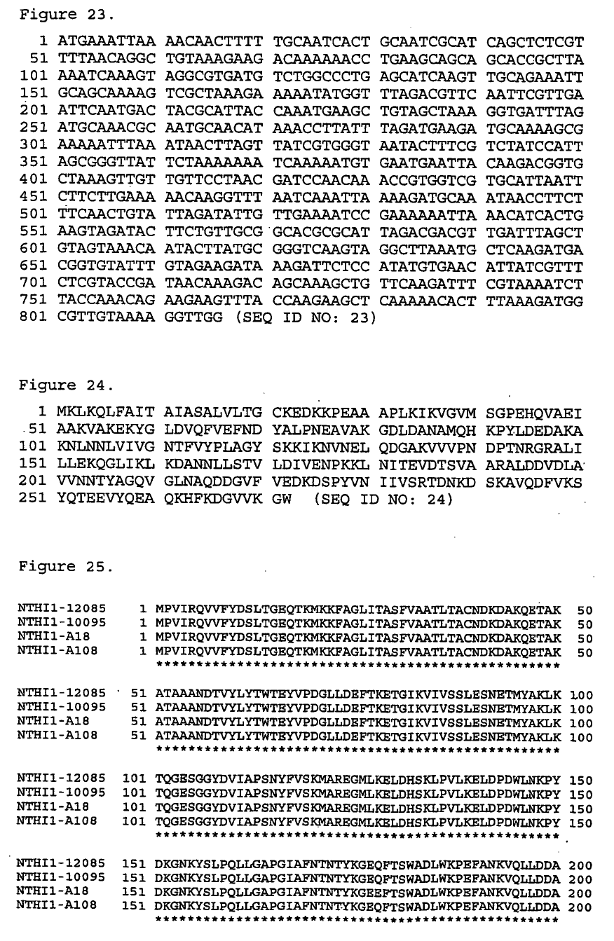

* Nucleotide position indicates the result of the cloning, i.e. the length ant the position in FIG. 1 of the PCR products.

### EXAMPLE 2

This example describes the PCR amplification and sequencing of BVH-NTHIL gene from other nontypeable H. influenzae strains and the evaluation of the level of molecular conservation of this gene.

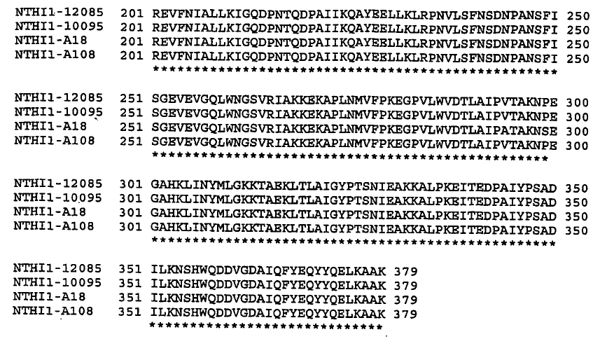

The respective coding regions of nontypeable H. influenzae gene BVH-NTHI1 from these strains were amplified by PCR (DNA Thermal Cycler GeneAmp PCR system 2400 Perkin Elmer, San Jose, Calif.) from genomic DNA using the following oligos: HAMJ342 (5′-CAAGGCGTTTTCATATGCCTGTCATTCGGCAGG-3′); HAMJ343 (5′-CTAATTGACCTCGAGTTTTGCTGCTTTTAATTCTTGATAATATTG-3′). PCR products were purified from agarose gels using a QIAquick gel extraction kit from QIAgen following the manufacturer's instructions (Chatsworth, Calif.) and the DNA inserts were sequenced (Taq Dye Deoxy Terminator Cycle Sequencing kit, ABI, Foster City, Calif.). The length of PCR fragments generated for all these eight strains was identical. Pairwise comparisons of nucleotides and predicted amino acid sequences from four of these strains (12085, 10095, A18 and A108) revealed 99% identity as shown in FIG. 25.

### EXAMPLE 3

This example illustrates the production and purification of recombinant nontypeable H. influenzae BVH-NTHI1 polypeptide.

The recombinant pET21-b+ plasmid with BVH-NTHI1 gene corresponding to the SEQ ID NO: 1 was used to electrotransform (Gene Pulser II apparatus, BIO-RAD Labs, Mississauga, Canada) E. coli strain Tuner(DE3) [F— ompT, hsdS (r_{b},m_{b}), gal, dcn, lacYI (DE3)] (Novagen, Madison, Wis.). Within this E. coli strain, the T7 promotor controlling expression of the recombinant polypeptide is specifically recognized by the T7 RNA polymerase (present on the λDE3 prophage) whose gene is under the control of the lac promotor which is inducible by isopropyl-β-d-thio-galactopyranoside (IPTG). The transformant Tuner(DE3)/rpET21 was grown at 37° C. with agitation at 250 rpm in LB broth (peptone log/L, yeast extract 5 g/L, NaCl log/L) containing 100 μg/ml of carbenicillin (Sigma-Aldrich Canada Ltd., Oakville, Canada), until the A₆₀₀ reached a value of 0.5. In order to induce the production of nontypeable H. influenzae BVH-NTHI1-HiseTag recombinant polypeptide, the cells were incubated for 3 additional hours at 30° C. in presence of IPTG at a final concentration of 1 mM. Induced cells from a 500 ml culture were pelleted by centrifugation and frozen at −70° C.

The purification of the recombinant polypeptides from the soluble cytoplasmic fraction of IPTG-induced Tuner(DE3)/rpET21 was done by affinity chromatography based on the properties of the His•Tag sequence (6 consecutive histidine residues) to bind to divalent cations (Ni²⁺) immobilized on the His•Bind metal chelation resin. Briefly, the pelleted cells obtained from a 500 mL IPTG-induced culture was resuspended in lysis buffer (20 mM Tris, 500 mM NaCl, 5 mM imidazole, pH 7.9) containing 1 mM of phenylmethylsulfonyl fluoride (PMSF; Sigma), sonicated and centrifugated at 16,000×g for 30 min to remove debris. The supernatant was deposited on a Ni-NTA agarose column (QIAgen, Mississauga, Ontario, Canada). The nontypeable H. influenzae BVH-NTHI1-His•Tag recombinant polypeptide was eluted with 250 mM imidazole-500 mM NaCl-20 mM Tris pH 7.9. Removal of salt and imidazole from the sample was performed by dialysis against PBS at 4° C. The quantity of recombinant polypeptide obtained from the soluble fraction of E. coli was estimated by MicroBCA (Pierce, Rockford, Ill.).

### EXAMPLE 4

This example illustrates the analysis of the immunogenicity and the protective ability of mice against nontypeable H. influenzae infection following immunization with BVH-NTHI1.

Groups of 5 female BALB/c mice (Charles River, Ontario, Canada) were immunized intranasally three times at two-week intervals with 25 μg of affinity purified BVH-NTHI1-His•Tag recombinant polypeptide in presence of the E. coli heat-labile toxin adjuvant (LT; 1 μg; Cedarlane Laboratories Ltd, Hornby, Canada) or, as a control, with adjuvant alone in PBS. Blood samples were collected from the orbital sinus on the day prior to each immunization and 14 days following the third (day 42) injection. Antibody titers were determined by ELISA on heat-killed and outer membrane vesicules of the nontypeable H. influenzae strain 12085. The secondary antibody used was a goat anti-mouse IgG+IgM (H+L) (Fc specific) conjugated to alcaline phosphatase (Jackson Immunoresearch Labs, Mississauga, Ontario). The reactive titer of an antiserum was defined as the reciprocal of the dilution showing a two-fold increase in absorbance over that obtained with the pre-immune serum sample. To investigate whether immune responses elicited by BVH-NTHI1 were functionally significant, in vivo protective efficacy was evaluated 14 days later in mice challenged intrapulmonarily with approximately 2×10⁵ CFU of the nontypeable H. influenzae strain 12085. Samples of the nontypeable H. influenzae challenge inoculum were plated on chocolate agar plates to verify the challenge dose. To measure the effectiveness of vaccination in limiting in vivo growth of nontypeable H. influenzae, mice were killed by an intraperitoneal injection of sodium pentobarbital (Euthanyl™) 5 h after infection. Bronchoalveolar lavages were assessed for bacterial clearance by plating of serial dilutions for bacterial count determination. Mice injected with adjuvant only were used as negative controls.

Results of the immunogenicity study (Table 2) showed that the level of specific antibodies in serum of immunized animals rose by around 1000-fold relative to control serum. The mean titer measured on OMP and heat-killed samples diverged by only one dilution, confirming that the BVH-NTHI1 polypeptide really represents a surface-exposed antigen.

TABLE 2
Effect of systemic immunization on NTHI-directed
antibody levels in serum^{a}

ELISA titer

| NTHI 12085 | Control | Immune |
|---|---|---|
| Outer membrane | 200 | 184 320 |
| polypeptides | | |
| Heat-killed | 360 | 368 640 |

^{a}Serum samples were collected from immune and control mice on day 42 of the immunization regimen.

Results of the protection study (Table 3) showed that strain 12085 readily multiplied in the lungs of the control animals such that by 5 hours postchallenge, the number of viable nontypeable H. influenzae cells had increased by 200%. In contrast, substantial pulmonary clearance of strain 12085 by the BVH-NTHI1-immunized mice was clearly evident, whereas only 35% of the original number of organisms deposit in the lungs of the immune animals were recovered by 5-hour postchallenge.

TABLE 3
Effect of systemic immunization on pulmonary clearance
of nontypeable H. influenzae 12085^{a}.

| | Nontypeable H. influenzae 12085 | |
|---|---|---|
| | (×10⁴) CFU | |
| BALB/c Mice | 0 h | 5 h |
| Control | 4.6 | 9.2 |
| Immunized | nd | 3.2^{b} |

^{a}Each value represents a mean of five animals at each time.
^{b}**P < 0.0098 calculated according to the unpaired t test with Welch's correction and compared to control.

### EXAMPLE 5

This example illustrates the recognition of recombinant polypeptides from nontypeable H. influenzae strain 12085 by human sera and by antisera from protected mice.

A standard Western blot technique was used to investigate whether purified recombinant polypeptides were recognized by human sera. In addition, antisera from mice immunized with outer membrane proteins preparations were tested against the purified recombinant polypeptides. Mice immunized with these preparations demonstrated increased pulmonary clearance following a challenge with nontypeable H. influenzae strain 12085. For the preparation of antigens, purified recombinant polypeptides were treated with sample buffer containing SDS and 2-mercaptoethanol for 5 min at 100° C. Polypeptides were resolved by SDS-PAGE according to the method of Laemmli, U.K. (1970) Cleavage of structural proteins during the assembly of the head of the bacteriophage T4. Nature, 227:680-685. After SDS-PAGE, the polypeptides were transferred electrophoretically from the gel to nitrocellulose paper by the method (Towbin, H., Staehelin, T. and Gordon, J. (1979) Electrophoretic transfer of protein from polyacrylamide gels to nitrocellulose sheets: procedure and some applications. Proc. Natl. Acad. Sci. USA, 76:4350-4354) and probed with human or mouse antisera. The detection of antigens reactive with the antibodies was performed by indirect enzyme-immunoassay using conjugated anti-mouse or anti-human immunoglobulins and a color substrate. Results in Table 4 show that most polypeptides have already been seen by the human immune system. As vaccine candidates, these polypeptides thus have a potential to induce a strong immune response in humans. Moreover, most polypeptides were recognized by sera from protected mice. The serum reactivities correlate to increased pulmonary clearance in the mouse model of infection.

TABLE 4
Reactivities of normal human sera and antisera from
protected mice with nontypeable H. influenzae 12085 recombinant
polypeptides.

| | | Pulmonary |
|---|---|---|
| Seq. | Serum reactivity^{a} | clearance |

| ID. | Polypeptide | Human | Mouse | (%)^{b} |
|---|---|---|---|---|
| 2 | BVH-NTHI1 | ++ | +++ | 65 |
| 4 | BVH-NTHI2 | + | +++ | 36 |
| 6 | BVH-NTHI3 | + | ++ | 56 |
| 8 | BVH-NTHI4 | ++ | +++ | 98 |
| 10 | BVH-NTHI5 | − | ++ | nd^{c} |
| 16 | BVH-NTHI8 | − | + | nd |
| 18 | BVH-NTHI9 | ++ | ++ | nd |
| 20 | BVH-NTHI10 | +++ | +++ | 44 |
| 24 | BVH-NTHI12 | ++++ | − | 20 |

^{a}The number of +'s indicates the reaction intensity The <<−>> sign indicates No reaction
^{b}Mice were immunised with the respective recombinant polypeptides and pulmonary clearance was measured as in Example 4
^{c}nd, not done

What is claimed is:

1. An isolated polynucleotide comprising a polynucleotide chosen from;
(a) a polynucleotide encoding a polypeptide having at least 70% identity to a second polypeptide comprising a sequence chosen from: SEQ ID NOs: 2, 4, 6, 8, 10, 12, 14, 16, 18, 20, 22 and 24 or fragments or analogs thereof;
(b) a polynucleotide encoding a polypeptide having at least 95% identity to a second polypeptide comprising a sequence chosen from: SEQ ID NOs: 2, 4, 6, 8, 10, 12, 14, 16, 18, 20, 22 and 24 or fragments or analogs thereof;
(c) a polynucleotide encoding a polypeptide comprising a sequence chosen from: SEQ ID NOs: 2, 4, 6, 8, 10, 12, 14, 16, 18, 20, 22 and 24 or fragments or analogs thereof;
(d) a polynucleotide encoding a polypeptide capable of generating antibodies having binding specificity for a polypeptide having a sequence chosen from SEQ ID NOs: 2, 4, 6, 8, 10, 12, 14, 16, 18, 20, 22 and 24 or fragments or analogs thereof;
(e) a polynucleotide encoding an epitope bearing portion of a polypeptide having a sequence chosen from SEQ ID NOs: 2, 4, 6, 8, 10, 12, 14, 16, 18, 20, 22 and 24 or fragments or analogs thereof;
(f) a polynucleotide complementary to a polynucleotide in (a), (b), (c), (d) or (e).

2. An isolated polynucleotide comprising a polynucleotide chosen from;
(a) a polynucleotide encoding a polypeptide having at least 70% identity to a second polypeptide comprising a sequence chosen from: SEQ ID NOs: 2, 4, 6, 8, 10, 12, 14, 16, 18, 20, 22 and 24;
(b) a polynucleotide encoding a polypeptide having at least 95% identity to a second polypeptide comprising a sequence chosen from: SEQ ID NOs: 2, 4, 6, 8, 10, 12, 14, 16, 18, 20, 22 and 24;
(c) a polynucleotide encoding a polypeptide comprising a sequence chosen from: SEQ ID NOs: 2, 4, 6, 8, 10, 12, 14, 16, 18, 20, 22 and 24;
(d) a polynucleotide encoding a polypeptide capable of generating antibodies having binding specificity for a polypeptide having a sequence chosen from SEQ ID NOs: 2, 4, 6, 8, 10, 12, 14, 16, 18, 20, 22 and 24;
(e) a polynucleotide encoding an epitope bearing portion of a polypeptide having a sequence chosen from SEQ ID NOs: 2, 4, 6, 8, 10, 12, 14, 16, 18, 20, 22 and 24;
(f) a polynucleotide complementary to a polynucleotide in (a), (b), (c), (d) or (e).

3. The polynucleotide of claim 1, wherein said polynucleotide is DNA.

4. The polynucleotide of claim 2, wherein said polynucleotide is DNA.

5. The polynucleotide of claim 1, wherein said polynucleotide is RNA.

6. The polynucleotide of claim 2, wherein said polynucleotide is RNA.

7. The polynucleotide of claim 1 that hybridizes under stringent conditions to either
(a) a DNA sequence encoding a polypeptide or
(b) the complement of a DNA sequence encoding a polypeptide;
wherein said polypeptide comprises SEQ ID NO: 2, 4, 6, 8, 10, 12, 14, 16, 18, 20, 22, 24, or fragments or analogs thereof.

8. The polynucleotide of claim 1 that hybridizes under stringent conditions to either
(a) a DNA sequence encoding a polypeptide or
(b) the complement of a DNA sequence encoding a polypeptide;
wherein said polypeptide comprises SEQ ID NO: 2, 4, 6, 8, 10, 12, 14, 16, 18, 20, 22, 24.

9. The polynucleotide of claim 1 that hybridizes under stringent conditions to either
(a) a DNA sequence encoding a polypeptide or
(b) the complement of a DNA sequence encoding a polypeptide;
wherein said polypeptide comprises at least 10 contiguous amino acid residues from a polypeptide comprising SEQ ID NO: 2, 4, 6, 8, 10, 12, 14, 16, 18, 20, 22, 24 or fragments or analogs thereof.

10. The polynucleotide of claim 1 that hybridizes under stringent conditions to either
(a) a DNA sequence encoding a polypeptide or
(b) the complement of a DNA sequence encoding a polypeptide;
wherein said polypeptide comprises at least 10 contiguous amino acid residues from a polypeptide comprising SEQ ID NO: 2, 4, 6, 8, 10, 12, 14, 16, 18, 20, 22, 24.

11. A vector comprising the polynucleotide of claim 1, wherein said DNA is operably linked to an expression control region.

12. A vector comprising the polynucleotide of claim 2, wherein said DNA is operably linked to an expression control region.

13. A host cell transfected with the vector of claim 11.

14. A host cell transfected with the vector of claim 12.

15. A process for producing a polypeptide comprising culturing a host cell according to claim 13 under conditions suitable for expression of said polypeptide.

16. A process for producing a polypeptide comprising culturing a host cell according to claim 14 under conditions suitable for expression of said polypeptide.

17. An isolated polypeptide comprising a member chosen from:
(a) a polypeptide having at least 70% identity to a second polypeptide having an amino acid sequence chosen from: SEQ ID NOs: 2, 4, 6, 8, 10, 12, 14, 16, 18, 20, 22 and 24 or fragments or analogs thereof;
(b) a polypeptide having at least 95% identity to a second polypeptide having an amino acid sequence chosen from: SEQ ID NOs: 2, 4, 6, 8, 10, 12, 14, 16, 18, 20, 22 and 24 or fragments or analogs thereof;
(c) a polypeptide comprising a sequence chosen from: SEQ ID NOs: 2, 4, 6, 8, 10, 12, 14, 16, 18, 20, 22 and 24 or fragments or analogs thereof;
(d) a polypeptide capable of generating antibodies having binding specificity for a polypeptide having a sequence chosen from SEQ ID NOs: 2, 4, 6, 8, 10, 12, 14, 16, 18, 20, 22 and 24 or fragments or analogs thereof;
(e) an epitope bearing portion of a polypeptide having a sequence chosen from SEQ ID NOs: 2, 4, 6, 8, 10, 12, 14, 16, 18, 20, 22 and 24 or fragments or analogs thereof;
(f) the polypeptide of (a), (b), (c), (d) or (e) wherein the N-terminal Met residue is deleted;
(g) the polypeptide of (a), (b), (c), (d) or (e) wherein the secretory amino acid sequence is deleted.

18. An isolated polypeptide comprising a member chosen from:
(a) a polypeptide having at least 70% identity to a second polypeptide having an amino acid sequence chosen from: SEQ ID NOs: 2, 4, 6, 8, 10, 12, 14, 16, 18, 20, 22 and 24;
(b) a polypeptide having at least 95% identity to a second polypeptide having an amino acid sequence chosen from: SEQ ID NOs: 2, 4, 6, 8, 10, 12, 14, 16, 18, 20, 22 and 24;
(c) a polypeptide comprising a sequence chosen from: SEQ ID NOs: 2, 4, 6, 8, 10, 12, 14, 16, 18, 20, 22 and 24;
(d) a polypeptide capable of generating antibodies having binding specificity for a polypeptide having a sequence chosen from SEQ ID NOs: 2, 4, 6, 8, 10, 12, 14, 16, 18, 20, 22 and 24;
(e) an epitope bearing portion of a polypeptide having a sequence chosen from SEQ ID NOs: 2, 4, 6, 8, 10, 12, 14, 16, 18, 20, 22 and 24;
(f) the polypeptide of (a), (b), (c), (d) or (e) wherein the N-terminal Met residue is deleted;
(g) the polypeptide of (a), (b), (c), (d) or (e) wherein the secretory amino acid sequence is deleted.

19. A chimeric polypeptide comprising two or more polypeptides having a sequence chosen from SEQ ID NOs: 2, 4, 6, 8, 10, 12, 14, 16, 18, 20, 22 and 24 or fragments or analogs thereof; provided that the polypeptides are linked as to formed a chimeric polypeptide.

20. A chimeric polypeptide comprising two or more polypeptides having a sequence chosen from SEQ ID NOs: 2, 4, 6, 8, 10, 12, 14, 16, 18, 20, 22 and 24 or fragments or analogs thereof; provided that the polypeptides are linked as to formed a chimeric polypeptide.

21. A pharmaceutical composition comprising a polypeptide according to any one of claims 17 to 20 and a pharmaceutically acceptable carrier, diluent or adjuvant.

22. A method for prophylactic or therapeutic treatment of otitis media, sinusitis, bronchitis, pneumonia and meningitis and bacteremia comprising administering to said individual a therapeutic or prophylactic amount of a composition according to claim 21.

23. A method for prophylactic or therapeutic treatment of Haemophilus influenzae bacterial infection in an individual susceptible to Haemophilus influenzae infection comprising administering to said individual a therapeutic or prophylactic amount of a composition according to claim 21.

24. The method of claim 23 wherein Haemophilus influenzae is Nontypeable Haemophilus influenzae.

25. The method of claim 23 wherein Haemophilus influenzae is Typeable Haemophilus influenzae.

26. A method for diagnostic of otitis media, sinusitis, bronchitis, pneumonia and meningitis and bacteremia comprising administering to said individual a therapeutic or prophylactic amount of a composition according to claim 21.

27. A method for diagnostic of Haemophilus influenzae bacterial infection in an individual susceptible to Haemophilus influenzae infection comprising administering to said individual a therapeutic or prophylactic amount of a composition according to claim 21.

28. The method of claim 27 wherein Haemophilus influenzae is Nontypeable Haemophilus influenzae.

29. The method of claim 27 wherein Haemophilus influenzae is Typeable Haemophilus influenzae.

30. Use of a pharmaceutical composition according to claim 21 for the prophylactic or therapeutic treatment of Haemophilus infection comprising administering to said individual a prophylactic or therapeutic amount of the composition.
